(19) United States
(12) Patent Application Publication
(10) Pub. No.: US 2004/0157902 A1
(43) Pub. Date: Aug. 12, 2004

(54) Integrin antagonists

(76) Inventors: Wolfgang Stähle, Ingelheim (DE); Alfred Jonczyk, Darmstadt (DE); Oliver Schadt, Rodenbach (DE); Simon Goodman, Griesheim (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/479,060

(22) PCT Filed: May 3, 2002
(86) PCT No.: PCT/EP02/04856

(30) Foreign Application Priority Data
Jun. 2, 2001 (DE) 101 27 041.0

(51) Int. Cl. A61K 31/41; A61K 31/325

(52) U.S. Cl. 514/381; 514/485; 548/256; 560/12

(57) ABSTRACT

Novel biphenyl derivatives of the general formula (I), in which A, X, R¹, R^{1′}, R^{1″}, R² R^{2′}, R^{2″} and n are as defined in Patent claim 1, stereoisomers thereof and physiologically acceptable salts or solvates thereof are novel inhibitors of integrin receptors, in particular of the αᵥβ₃, αᵥβ₅ and/or αᵥβ₆ integrin receptors. The novel compounds can be used as medicaments.

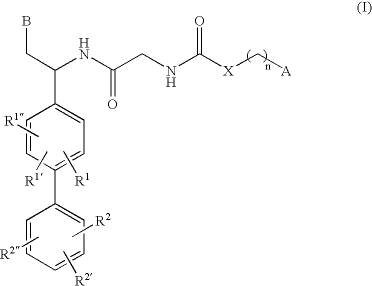

The invention relates to novel compounds of the formula I

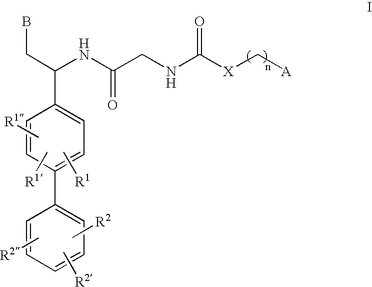

in which

A is NH₂, —(HN═)C—NH₂, —NH—C(═NH)—NH₂, A′-C(═NH)—NH—, Het¹- or Het¹-NH—, where the primary amino groups may also be provided with conventional amino-protecting groups,

B is tetrazolyl or an alkylsulfonylaminocarbonyl group,

R is H, A′, C₃-C₁₄-cycloalkyl, C₆-C₁₀-aryl or C₇-C₁₄-aralkyl, which may be monosubstituted or polysubstituted by R³ and whose alkyl carbon chain may be interrupted by O,

R¹, R^{1′}

and R^{1″}, independently of one another, are H, F, Cl, Br, I, NO₂, NH₂, NHR, NRR, OH, OR, CO—R, SO₃R, SO₂R or SR,

R², R^{2′} and R^{2″}, independently of one another, are H, F, Cl, Br, I, NO₂, NH₂, NHR, NRR, OH, OR, CO—R, SO₃R, SO₂R or SR,

R³ is F, Cl, Br, I, NO₂, CF₃, OH, CN, OCF₃, SCF₃, methoxy or ethoxy,

Het¹ is a monocyclic or bicyclic heterocyclic radical having from 1 to 4 N atoms, which may be unsubstituted or monosubstituted or disubstituted by A′, NHA′, NA′₂ and/or NH₂,

A′ is alkyl having from 1 to 8 carbon atoms,

x is nothing, O, NH or CH₂

n is 2, 3 or 4,

stereoisomers thereof and physiologically acceptable salts and solvates thereof.

Compounds having a partially similar structure are disclosed in WO 96/22966 A1, WO 97/08145 A1 and WO 00/48996 A2, where all compounds are effective as integrin receptor inhibitors. Integrins are membrane-bound, heterodimeric glycoproteins which consist of an □-subunit and a smaller β-subunit. The relative affinity and specificity for ligand binding is determined by the combination of the different α- and β-subunits. According to the disclosure content of the said patent applications, the compounds of WO 96/22966 A1 selectively inhibit the α₄β₁ integrin receptor, and the compounds of WO 97/08145 A1 selectively inhibit the αᵥβ₃ integrin receptor. The compounds of WO 00/48996 A2 inhibit principally αᵥβ₃ and αᵥβ₅ integrin receptors.

The invention had the object of finding novel compounds having valuable properties, in particular those which are used for the preparation of medicaments.

It has been found that the compounds of the formula I, stereoisomers thereof and salts thereof have very valuable pharmacological properties and are well tolerated. In particular, the compounds are distinguished by very high efficiency. They act as antagonists of integrin receptors, in particular of the αᵥβ₃, αᵥβ₅ and/or αᵥβ₆ integrin receptors. In addition, they octanol/water have very favourable partition coefficients in octanol/water (logD values).

If an active ingredient is introduced into an octanol/water mixture, it partitions between the two phases at a given pH in accordance with its lipophilicity/hydrophilicity. The ratio of the partitioning of the active ingredient between the octanol and water phases is known as the partition coefficient. Absorption of active ingredients requires that they both dissolve in aqueous media and also pass through the corresponding membranes. For the latter, a certain lipophilicity of the active ingredient is necessary. The requisite lipophilicity arises from the lipophilicity of the respective membrane. Thus, absorption in the intestine requires a logD value of >−1.50 and passage through the blood-brain barrier requires a logD value of >0.5 (Lipinski C. A: Adv. Drug Del. Rev. 23 (1997), 3-25). A favourable logD value is an essential prerequisite for absorption of an active ingredient.

The integrins are ascribed various physiological and pathological functions, which are revealed in detail, for example, by the following review papers: Integrins and signal transduction. Dedhar-S, Curr-Opin-Hematol. 1999 January; 6(1): 37-43, Integrins take partners: cross-talk between integrins and other membrane receptors. Porter-J C; Hogg-N, Trends-Cell-Biol. 1998 October; 8(10): 390-6, Regulation of integrin-mediated adhesion during cell migration. Cox-E A; Huttenlocher-A, Microsc-Res-Tech. 1998 Dec. 1; 43(5): 412-9, The role of integrins in the malignant phenotype of gliomas. Uhm-J H; Gladson-C L; Rao-J S, Front-Biosci. 1999 Feb. 15; 4: D188-99, or Sperm disintegrins, egg integrins, and other cell adhesion molecules of mammalian gamete plasma membrane interactions. Evans-JP Front-Biosci. 1999 Jan. 15; 4: DI 14-31.

An important role here is ascribed to the αᵥ integrins, as described, for example, in The role of alpha v-integrins in tumour progression and metastasis. Marshall-J F; Hart-I R Semin-Cancer-Biol. 1996 June; 7(3): 129-38 or The role of alpha v-integrins during angiogenesis. Eliceiri-B P and Cheresh-D A Molecular Medicine 4: 741-750 (1998).

These integrins also include αᵥβ₆ Epithelial integrins. Sheppard-D Bio-essays. 1996 August; 18(8): 655-60 and the two integrins αᵥβ₃ and αᵥβ₅ which are known adhesion receptors, whose biological importance has been referred to, for example, in J. A. Varner et al. Cell Adhesion and Communication 3, 367-374 (1995) and in J. Samanen et al. Curr. Pharmaceutical Design, 3, 545-584 (1997).

αᵥβ₆ is a relatively rare integrin (Busk et al., 1992 J. Biol. Chem. 267(9), 5790), which is increasingly formed in epithelial tissue during repair processes and preferentially binds the natural matrix molecules fibronectin and tenascin (Wang et al., 1996, Am. J. Respir. Cell Mol. Biol. 15(5), 664). The physiological and pathological functions of αᵥβ₆ are still not known precisely, but it is assumed that this integrin plays an important role in physiological processes and disorders (for example inflammation, wound healing and tumours) in which epithelial cells are involved. Thus, αᵥβ₆ is expressed on keratinocytes in wounds (Haapasalmi et al., 1996, J. Invest. Dermatol. 106(1), 42), from which it can be assumed that, besides wound-healing processes and inflammation, other pathological occurrences in the skin, such as, for example, psoriasis, can be influenced by agonists or antagonists of the said integrin. Furthermore, αᵥβ₆ plays a role in the respiratory tract epithelium (Weinacker et al., 1995, Am. J. Respir. Cell Mol. Biol. 12(5), 547, and consequently corresponding agonists/antagonists of this integrin could successfully be employed in respiratory tract disorders, such as bronchitis, asthma, lung fibrosis and respiratory tract tumours. Finally, it is known that αᵥβ₆ also plays a role in the intestinal epithelium, and consequently corresponding integrin agonists/antagonists could be used in the treatment of inflammation, tumours and wounds of the stomach/intestinal tract. Microorganisms and viruses also have integrin receptors, in particular αᵥβ₆ receptors. For example, αᵥβ₅ receptors are coreceptors for adeno-viruses or αᵥβ₅/αᵥβ₅ receptors are coreceptors for HIV. The integrin antagonists/agonists could therefore also be employed for the treatment of infections, in particular viral infections.

The effect of a compound on an αᵥβ₆ integrin receptor and thus the activity as an inhibitor can be demonstrated, for example, by the method described by J. W. Smith et al. in J. Biol. Chem. 1990, 265, 12267-12271.

Besides the preferred inhibition of αᵥβ₆ integrin receptors, the compounds also act as inhibitors of αᵥβ₃ or αᵥβ₅ integrin receptors and as inhibitors of glycoprotein IIb/IIIa. Integrin αᵥβ₃, for example, is expressed on a number of cells, for example endothelial cells, cells of the smooth vascular muscles, for example of the aorta, cells for the breakdown of bone matrix (osteoclasts) or tumour cells.

The action of the compounds according to the invention can be demonstrated, for example, by the method described by J. W. Smith et al. in J. Biol. Chem. 1990, 265, 12267-12271.

The dependence of the occurence of angiogenesis on the interaction between vascular integrins and extracellular matrix proteins has been described by P. C. Brooks, R. A. Clark and D. A. Cheresh in Science 1994, 264, 569-571.

The possibility of inhibiting this interaction and so initiating apoptosis (programmed cell death) of angiogenic vascular cells by a cyclic peptide has been described by P. C. Brooks, A. M. Montgomery, M. Rosenfeld, R. A. Reisfeld, T. Hu, G. Klier and D. A. Cheresh in Cell 1994, 79, 1157-1164. In this, for example, αᵥβ₃ antagonists or antibodies against αᵥβ₃ were described which cause shrinkage of tumours due to the initiation of apoptosis.

The experimental evidence that the compounds according to the invention also prevent the attachment of living cells to the corresponding matrix proteins and accordingly also prevent the attachment of tumour cells to matrix proteins can be provided in a cell adhesion test analogously to the method of F. Mitjans et al., J. Cell Science 1995, 108, 2825-2838.

The compounds of the formula I are able to inhibit the binding of metallo-proteinases to integrins and thus prevent the cells utilising the enzymatic activity of the proteinase. An example can be found in the ability of a cyclo-RGD peptide to inhibit the binding of MMP-2 (matrix-metallo-proteinase-2) to the vitronectin receptor αᵥβ₃, as described in P. C. Brooks et al., Cell 1996, 85, 683-693.

Compounds of the formula I which block the interaction of integrin receptors and ligands, such as, for example, of fibrinogen to the fibrinogen receptor (glycoprotein IIb/IIIa), prevent, as antagonists, the spread of tumour cells by metastasis and can therefore be employed as antimetastatic substances in operations in which tumours are removed or attacked surgically. This is confirmed by the following observations:

The spread of tumour cells from a local tumour into the vascular system occurs through the formation of microaggregates (microthromboses) due to the interaction of the tumour cells with blood platelets. The tumour cells are masked by the protection in the microaggregate and are not recognised by the immune system cells. The microaggregates are able to attach to vessel walls, simplifying further penetration of tumour cells into the tissue. Since the formation of microthromboses is promoted by ligand binding to the corresponding integrin receptors, for example αᵥβ₃ or α_{IIb}β₃, on activated blood platelets, the corresponding antagonists can be regarded as effective metastasis inhibitors.

The action of a compound on an αᵥβ₅ integrin receptor and thus the activity as an inhibitor can be demonstrated, for example, by the method described by J. W. Smith et al. in J. Biol. Chem. 1990, 265, 12267-12271.

The compounds can be administered to humans or animals locally or systemically, orally, intravenously, intraperitoneally, intramuscularly, subcutaneously, transdermally, nasally, buccally or iontophoretically.

A measure of the uptake of a medicament active ingredient in an organism is its bioavailability.

If the medicament active ingredient is administered to the organism intravenously in the form of an injection solution, its absolute bioavailability, i.e. the proportion of the pharmaceutical species which is unchanged in the systemic blood, i.e. enters the general circulation, is 100%.

On oral administration of a therapeutic active ingredient, the active ingredient is generally in the form of a solid in the formulation and must therefore first dissolve in order that it can overcome the entry barriers, for example the gastrointestinal tract, the oral mucous membrane, nasal membranes or the skin, in particular the stratum corneum, and can be absorbed by the body. Pharmacokinetic data, i.e. on the bioavailability, can be obtained analogously to the method of J. Shaffer et al., J. Pharm. Sciences, 1999, 88, 313-318.

As described above, a measure that can be used for the absorbability of an active ingredient is its logD value.

The compounds of the formula I have at least one centre of chirality and can therefore occur in a number of stereoisomeric forms. All of these forms (for example D and L forms) and mixtures thereof (for example the DL forms) are included in the formula.

The compounds according to the invention according to claim 1 also include so-called prodrug derivatives, i.e. compounds of the formula I modified with, for example, alkyl or acyl groups, sugars or oligopeptides, which are rapidly cleaved in the organism to give the effective compounds according to the invention.

Furthermore, free amino groups or free hydroxyl groups can be provided as substituents of compounds of the formula I with corresponding protecting groups.

The term solvates of the compounds of the formula I is taken to mean adductions of inert solvent molecules onto the compounds of the formula I which form owing to their mutual attractive force. Solvates are, for example, mono- or dihydrates or addition compounds with alcohols, such as, for example, with methanol or ethanol.

The invention relates to the compounds of the formula II

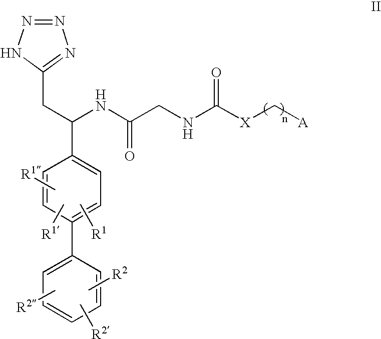

according to the formula I, salts and solvates thereof, and to a process for the preparation of compounds of the formula II and salts and solvates thereof, in which A, X, R¹, R^{1′}, R^{1″}, R², R²′, R^{2″} and n are as defined in the formula I, characterised in that

(a) a compound of the formula III

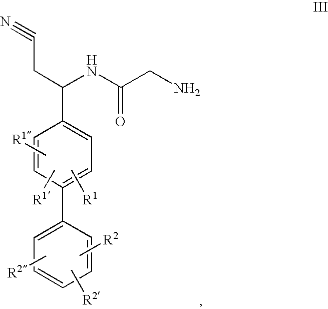

in which R¹, R^{1′}, R^{1″}, R², R^{2′} and R^{2″} are as defined in the formula I and in which, in the case where R¹, R^{1′}, R^{1″}, R², R^{2′} and/or R^{2″} contain free hydroxyl and/or amino groups, these are protected by a protecting group,

is reacted with a compound of the formula IV

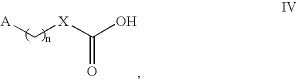

in which A and n are as defined in the formula I, and X is nothing or —(CH₂)—, and in which, in the case where A contains free amino groups, these are each protected by protecting groups,

to give a compound of the general formula V

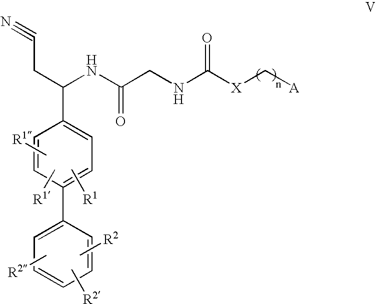

in which A, R¹, R^{1′}, R^{1″}, R², R^{2′}, R^{2″} and n are as defined in the formula I and X is —(CH₂)—,

and the resultant compound of the formula V is subsequently converted into a compound of the above formula II, in which R¹, R^{1′}, R^{1″}, R², R^{2′}, R^{2″}, A and n are as defined therein, and X is —(CH₂)—, and, if desired, the protecting groups present on A, R¹, R^{1′}, R^{1″}, R², R^{2′} and/or R^{2″} are removed, or

(b) a compound of the formula VI

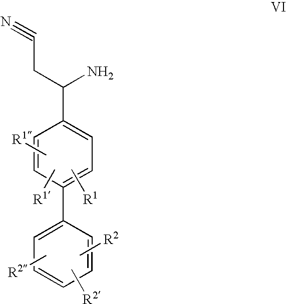

in which R¹, R^{1′}, R^{1″}, R², R^{2′} and R^{2″} are as defined in the formula I and in which, in the case where R¹, R^{1′}, R^{1″}, R², R^{2′} and/or R^{2″} contain free hydroxyl and/or amino groups, these are protected by a protecting group,

is reacted with a compound of the formula VII

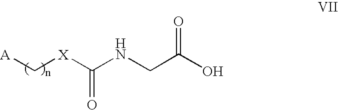

in which A, X and n are as defined in the formula I and in which, in the case where A contains free amino groups, these are each protected by protecting groups,

to give a compound of the above general formula V, in which R¹, R^{1′}, R^{1″}, R², R^{2′}, R^{2″}, A and n are as defined therein, and X is as defined in the formula I,

and the resultant compound of the formula V is subsequently converted into a compound of the above general formula II, in which R¹, R^{1′}, R^{1″}, R², R^{2′}, R^{2″}, A, X and n are as defined therein,

and, if desired, the protecting groups present on A, R¹, R^{1′}, R^{1″}, R², R^{2′} and/or R^{2″} are removed, or

(c) one or more of the radicals R¹, R^{1′}, R^{1″}, R², R^{2′} and/or R^{2″} in a compound of the formula II, in which R¹, R^{1′}, R^{1″}, R², R^{2′}, R^{2″}, A, X and n are as defined therein, are converted into one or more radicals R¹, R^{1′}, R^{1″}, R², R^{2′} and/or R^{2″} by, for example,

i) alkylating a hydroxyl group or

ii) alkylating an amino group, and/or

a basic or acidic compound of the formula II is converted into one of its salts or solvates by treatment with an acid or base.

The invention furthermore relates to the compounds of the formula VIII

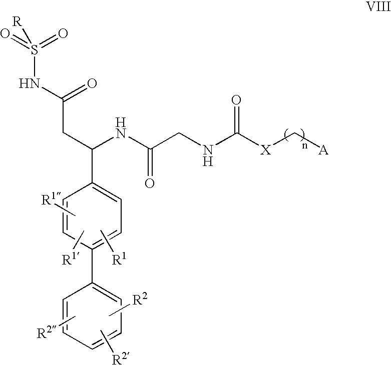

according to the formula I, salts and solvates thereof, and to a process for the preparation of compounds of the formula VIII and salts and solvates thereof, in which A, X, R, R¹, R^{1′}, R^{1″}, R², R^{2′}, R^{2″} and n are as defined in the formula I, characterised in that

(a) a compound of the formula IX

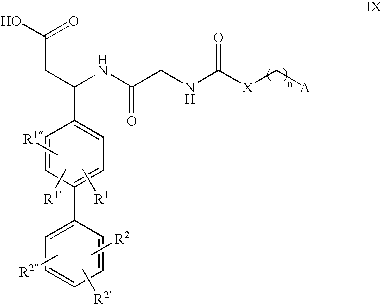

in which A, X, R¹, R^{1′}, R^{1″}, R², R^{2′}, R^{2″} and n are as defined in the formula I, and in which, in the case where A, R¹, R^{1′}, R^{1″}, R², R^{2′} and/or R^{2″} contain free hydroxyl or amino groups, these are protected by a protecting group,

is reacted with a compound of the formula X

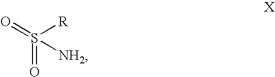

in which R is as defined in the formula I, to give a compound of the above-mentioned general formula VII, in which A, X, R, R¹, R^{1′}, R^{1″}, R², R^{2′}, R^{2″} and n are as defined therein, and, if desired, the protecting groups present on A′, R¹, R^{1′}, R^{1″}, R², R^{2′} and/or R^{2″} are removed, or

(b) one or more radicals R¹, R^{1′}, R^{1″}, R², R^{2′} and/or R^{2″} in a compound of the formula VII are converted into one or more radicals R¹, R^{1′}, R^{1″}, R², R^{2′} and/or R^{2″} by, for example,

i) alkylating a hydroxyl group or

ii) alkylating an amino group, and/or

a basic or acidic compound of the formula VIII is converted into one of its salts or solvates by treatment with an acid or base.

Throughout the invention, all radicals which occur more than once, such as, for example, A′, R¹, R^{1′}, R^{1″}, R², R^{2′}, R^{2″}, may be identical or different, i.e. are independent of one another.

In the above formulae, A′ is alkyl, is linear or branched, and has from 1 to 8, preferably 1, 2, 3, 4, 5 or 6 carbon atoms. A′ is preferably methyl, furthermore ethyl, n-propyl, isopropyl, n-butyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, heptyl or octyl. Further preferred embodiments of A′ are the said alkyl groups, which, however, may be monosubstituted or polysubstituted by Hal or NO₂, preferably trifluoromethyl, 2,2,2-trifluoroethyl or 2-nitroethyl, or alkyl groups, whose carbon chain may be interrupted by —O—, preferably —CH₂—O—CH₃, —CH₂—O—CH₂—CH₃ or —CH₂—CH₂—O—CH₃.

A′ is particularly preferably methyl or ethyl.

C₃-C₁₄-cycloalkyl has from 3 to 14 carbon atoms and is preferably cyclopropyl, cyclobutyl, cyclopentyl, cyclohexyl, cycloheptyl or cyclooctyl, particularly preferably cyclohexyl. Cycloalkyl is likewise a monocyclic or bicyclic terpene, preferably p-menthane, menthol, pinane, bornane or camphor, where each known stereoisomeric form is included, or adamantyl. For camphor, this is both L-camphor and D-camphor.

C₇-C₁₄-aralkyl is preferably benzyl, phenethyl, naphth-1-ylmethyl, naphth-2-ylmethyl, naphth-1-ylethyl or naphth-2-ylethyl, particularly preferably benzyl or phenethyl.

C₆-C₁₀-aryl is preferably unsubstituted or polysubstituted phenyl or naphthyl, in particular phenyl or naphthyl which is unsubstituted, monosubstituted, disubstituted or trisubstituted by A′, OH, OA′, NH₂, NHA′, NA′₂, NO₂, CF₃, CN, F, Cl, Br, I, CO-A′, SO₃A′, SO₂A′ or SA′.

Het¹ is an unsubstituted or substituted monocyclic or bicyclic aromatic heterocyclic ring system having 1, 2, 3 or 4, preferably 1 or 2, N atoms. Het¹ is preferably 1-, 2- or 3-pyrrolyl, 1-, 2-, 4- or 5-imidazolyl, 1-, 3-, 4- or 5-pyrazolyl, 2-, 3- or 4-pyridyl, 2-, 4-, -5- or 6-pyrimidinyl, furthermore preferably 1,2,3-triazol-1-, 4- or -5-yl, 1,2,4-triazol-1-, -3- or 5-yl, 1- or 5-tetrazolyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 1H-imidazo[4,5-b]pyridin-2-yl or 1,8-naphthyridin-7-yl, each of which is unsubstituted or monosubstituted or disubstituted by A′, NHA′ or NA′₂.

The heterocyclic radicals may also be partially or fully hydrogenated. Het¹ may thus also be, for example, 2,3-dihydro-1-, -2-, -3-, 4- or -5-pyrrolyl, 2,5-dihydro-1-, -2-, -3-, 4- or -5-pyrrolyl, 1-, 2- or 3-pyrrolidinyl, tetrahydro-1-, -2- or -4-imidazolyl, 4,5-dihydroimidazol-2-yl, 2,3-dihydro-1-, -2-, -3-, 4- or -5-pyrazolyl, tetrahydro-1-, -3- or 4-pyrazolyl, 1,4-dihydro-1-, -2-, -3- or 4-pyridyl, 1,2,3,4-tetrahydro-1-, -2-, -3-, 4-, -5- or -6-pyridyl, 1-, 2-, 3- or 4-piperidinyl, hexahydro-1-, -3- or 4-pyridazinyl, hexahydro-1-, -2-, 4- or -5-pyrimidinyl, 1-, 2- or 3-piperazinyl, 1,2,3,4-tetrahydro-1-, -2-, -3-, 4-, -5-, -6-, -7- or -8-quinolyl, 1,2,3,4-tetrahydro-1-, -2-, -3-, 4-, -5-, -6-, -7- or -8-isoquinolyl or 1,2,3,4-tetrahydro-1,8-naphthyridin-7-yl.

Hydrogenated or partially hydrogenated Het¹ radicals may additionally be substituted by ═NH or carbonyl oxygen.

Het¹ in A is preferably in the form of Het¹-NH. Particular preference is given here to

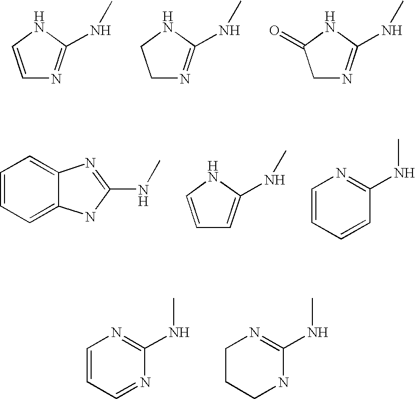

Very particular preference is given here to pyridin-2-ylamino.

R¹, R^{1′} and R^{1″} as well as R², R^{2′} and R^{2″} are preferably H, F, Cl, Br, I, NO₂, NH₂, NHA′, NA′₂, OA′, CO-A′, SO₃A′, SO₂A′ or SA′.

Amino-protecting group preferably means formyl, acetyl, propionyl, butyryl, phenylacetyl, benzoyl, tolyl, POA, methoxycarbonyl, ethoxycarbonyl, 2,2,2-trichloroethoxycarbonyl, BOC, 2-iodoethoxycarbonyl, CBZ (“carbobenzoxy”), 4-methoxybenzyloxycarbonyl, FMOC, Mtr or benzyl.

B is preferably tetrazol-5-yl or alkylsulfonylaminocarbonyl, in which the alkyl group is as defined for A′. Particular preference is given to methanesulfonylaminocarbonyl.

n is preferably 2, 3 or 4, and n is very particularly preferably 2 or 3.

“Poly” substituted means mono-, di-, tri- or tetrasubstituted.

Pol denotes a solid phase with no terminal functional group, as explained in greater detail below. The terms solid phase and resin are used synonymously below.

Accordingly, the invention relates in particular to the compounds of the formula I in which at least one of the said radicals has one of the preferred meanings indicated above. Some preferred groups of compounds may be expressed by the following sub-formulae Ia) to Ig), which conform to the formula I and in which the radicals not designated in greater detail have the meaning indicated under the formula I, but in which

in

Ia) A is —(HN═)C—NH₂, —NH—C(═NH)—NH₂, A′-C(═NH)—NH—, Het¹ or Het¹-NH—, where the primary amino groups may also be provided with conventional amino-protecting groups, and in which

Het¹ is a monocyclic or bicyclic, aromatic or partially or fully hydrogenated heterocyclic ring system having 1 or 2 N atoms which is unsubstituted or monosubstituted or disubstituted by A′, NHA′, NH₂ and/or NA′₂, in which, in the case of a hydrogenated or partially hydrogenated heterocyclic ring system, this may additionally be substituted by ═NH or carbonyl oxygen,

Ib) A is —(HN═)C—NH₂, —NH—C(═NH)—NH₂ or Het¹-NH—, where the primary amino groups may also be provided with conventional amino-protecting groups, and in which Het¹-NH— is

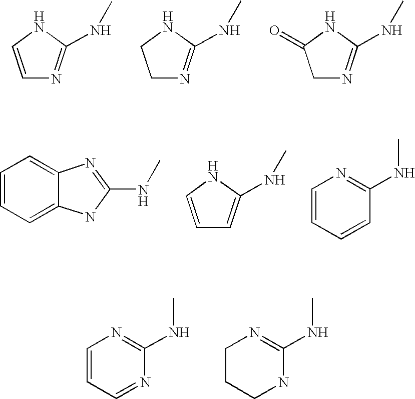

Ic) A is NH₂, —(HN═)C—NH₂, —NH—C(═NH)—NH₂, A′-C(═NH)—NH—, Het¹- or Het¹-NH—, where the primary amino groups may also be provided with conventional amino-protecting groups,

B is tetrazol-5-yl or an alkylsulfonylaminocarbonyl group,

R is H, A′, C₆-C₁₄-cycloalkyl or C₇-C₁₄-aralkyl, which may be monosubstituted or disubstituted by R³ and whose alkyl carbon chain may be interrupted by 0,

R¹, R^{1′} and R^{1″}, independently of one another, are H, F, Cl, Br, I, NO₂, NH₂, NHA′, NA′₂, OA′, CO-A′, SO₃A′, SO₂A′ or SA′,

R¹, R^{1′} and R^{1″}, independently of one another, are H, F, Cl, Br, I, NO₂, NH₂, NHA′, NA′₂, OA′, CO-A′, SO₃A′, SO₂A′ or SA′,

R³ is F, Cl, Br, I, NO₂, CF₃, OH, CN, OCF₃, SCF₃, methoxy or ethoxy,

Het¹-NH— is

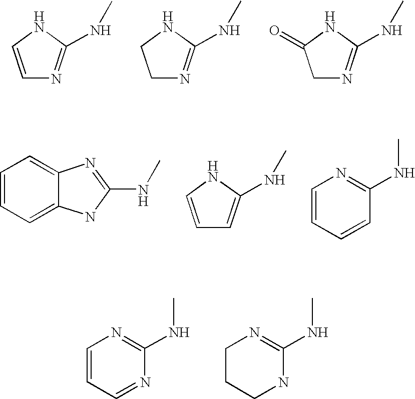

A′ is alkyl having from 1 to 6 carbon atoms,

X is nothing, O, NH or CH₂,

n is 2, 3 or 4,

Id) A is —(HN═)C—NH₂, —NH—C(═NH)—NH₂ or Het¹-NH—, where the primary amino groups may also be provided with conventional amino-protecting groups,

B is tetrazol-5-yl or an alkylsulfonylaminocarbonyl group, in which alkyl has 1, 2, 3, 4, 5 or 6 carbon atoms,

R is H, A′, cyclohexyl, benzyl or phenylethyl,

R¹, R^{1′} and R^{1″}, independently of one another, are H, F, Cl, Br, I, NO₂, NHA′, NA′₂ or OA′,

R², R^{2′} and R^{2″}, independently of one another, are H, F, Cl, Br, I, NO₂, NHA′, NA′₂ or OA′,

Het¹-NH— is

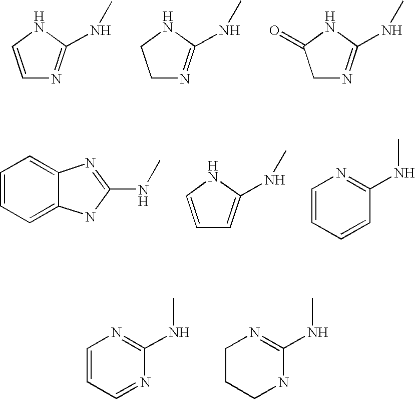

A is alkyl having 1, 2, 3, 4, 5 or 6 carbon atoms,

X is nothing, NH or CH₂,

n is 2, 3 or 4,

Ie) A is Het¹-NH— with

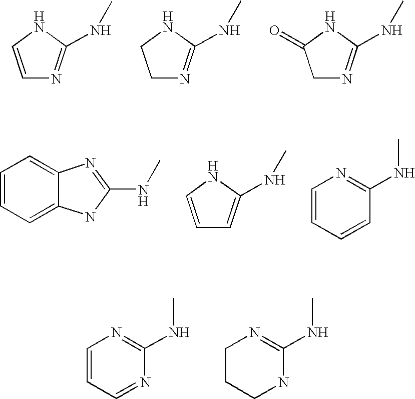

B is tetrazolyl or an alkylsulfonylaminocarbonyl group,

R is H, A′, cyclohexyl, benzyl or phenylethyl,

R¹, R^{1′} and R^{1″}, independently of one another, are H, F, Cl, Br, I, NO₂, NR₂, OR or CO—R,

R², R^{2′} and R^{2″}, independently of one another, are H, F, Cl, Br, I, NO₂, NR₂, OR or CO—R,

A′ is alkyl having from 1 to 8 carbon atoms,

X is nothing, O, NH or CH₂,

n is 2, 3 or 4,

If) A is Het¹-NH— with

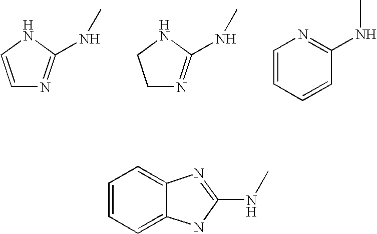

B is tetrazol-5-yl or an alkylsulfonylaminocarbonyl group, in which alkyl has 1, 2, 3, 4, 5 or 6 carbon atoms,

R¹, R^{1′} and R^{1″}, independently of one another, are H, F, Cl, Br, I, NO₂, NHA′, NA′₂, OA′ or CO-A′,

R², R^{2′} and R^{2″}, independently of one another, are H, F, Cl, Br, I, NO₂, NHA′, NA′₂, OA′ or CO-A′,

A′ is alkyl having from 1 to 6 carbon atoms,

X is nothing, O, NH or CH₂,

n is 2, 3 or 4,

Ig) A is Het¹-NH— with

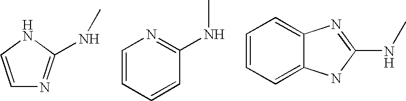

B is tetrazol-5-yl or an alkylsulfonylaminocarbonyl group, in which alkyl has 1, 2, 3 or 4 carbon atoms,

R¹, R^{1′} and R^{1″}, independently of one another, are H, F, Cl, Br, I, NO₂, NHA′, NA′₂ or OA′,

R², R^{2′} and R^{2″}, independently of one another, are H, F, Cl, Br, I, NO₂, NHA′, NA′₂ or OA′,

A′ is alkyl having from 1 to 4 carbon atoms,

X is nothing, O, NH or CH₂,

n is 2, 3 or 4.

Particular preference is given to the compounds of the general formula I mentioned below:

N-[1-biphenyl-4-yl-2-(1H-tetrazol-5-yl)ethyl]-2-[5-(pyridin-2-ylamino)-pentanoylamino]acetamide

N-[1-biphenyl-4-yl-2-(1H-tetrazol-5-yl)ethyl]-2-{3-[3-(pyridin-2-ylamino)propyl]ureido}acetamide

N-[1-biphenyl-4-yl-2-(methanesulfonylaminocarbonyl)ethyl]-2-[5-(pyridin-2-ylamino)pentanoylamino]acetamide.

The compounds of the formula I according to claim 1 and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for said reactions. Use can also be made here of variants which are known per se, but are not mentioned here in greater detail.

If desired, the starting materials can also be formed in situ, so that they are not isolated from the reaction mixture, but are instead immediately converted further into the compounds of the formula I according to claim 1.

It is also possible for a plurality of—identical or different—protected amino and/or hydroxyl groups to be present in the molecule of the starting material. If the protecting groups present differ from one another, they can in many cases be removed selectively (cf. in this respect: T. W. Greene, P. G. M. Wuts, Protective Groups in Organic Chemistry, 2nd Edn., Wiley, New York 1991 or P. J. Kocienski, Protecting Groups, 1st Edn., Georg Thieme Verlag, Stuttgart—New-York, 1994).

The term “amino-protecting group” is generally known and relates to groups which are suitable for protecting (blocking) an amino group against chemical reactions. Typical of such groups are, in particular, unsubstituted or substituted acyl, aryl, aralkoxymethyl or aralkyl groups. Since the amino-protecting groups are removed after the desired reaction (or synthesis sequence), their type and size is furthermore not crucial; however, preference is given to those having 1-20, in particular 1-8 carbon atoms. The term “acyl group” is to be understood in the broadest sense in connection with the present process. It includes acyl groups derived aliphatic, araliphatic, alicyclic, aromatic and heterocyclic carboxylic acids or sulfonic acids, as well as, in particular, alkoxycarbonyl, alkenyloxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as phenoxyacetyl; alkoxycarbonyl, such as methoxycarbonyl, ethoxycarbonyl, 2,2,2-trichloroethoxycarbonyl, BOC and 2-iodoethoxycarbonyl; alkenyloxycarbonyl, such as allyloxycarbonyl (Aloc), aralkoxycarbonyl, such as CBZ (synonymous with Z), 4-methoxybenzyloxycarbonyl (MOZ), 4-nitrobenzyloxycarbonyl and 9-fluorenylmethoxycarbonyl (Fmoc); 2-(phenylsulfonyl)ethoxycarbonyl; trimethylsilylethoxycarbonyl (Teoc), and arylsulfonyl, such as 4-methoxy-2,3,6-trimethylphenylsulfonyl (Mtr). Preferred amino-protecting groups are BOC, Fmoc and Aloc, furthermore CBZ, benzyl and acetyl. Particularly preferred protecting groups are BOC and Fmoc.

The term “hydroxyl-protecting group” is likewise generally known and relates to groups which are suitable for protecting a hydroxyl group against chemical reactions. Typical of such groups are the above-mentioned unsubstituted or substituted aryl, aralkyl, aroyl or acyl groups, furthermore also alkyl groups, alkyl-, aryl- and aralkylsilyl groups, and O,O- and O,S-acetals. The nature and size of the hydroxyl-protecting groups is not crucial since they are removed again after the desired chemical reaction or synthesis sequence; preference is given to groups having 1-20 carbon atoms, in particular 1-10 carbon atoms. Examples of hydroxyl-protecting groups are, inter alia, aralkyl groups, such as benzyl, 4-methoxybenzyl and 2,4-di-methoxybenzyl, aroyl groups, such as benzoyl and p-nitrobenzoyl, acyl groups, such as acetyl and pivaloyl, p-toluenesulfonyl, alkyl groups, such as methyl and tert-butyl, but also allyl, alkylsilyl groups, such as trimethylsilyl (TMS), triisopropylsilyl (TIPS), tert-butyldimethylsilyl (TBS) and triethylsilyl, trimethylsilylethyl, aralkylsilyl groups, such as tert-butyldiphenylsilyl (TBDPS), cyclic acetals, such as isopropylidene acetal, cyclopentylidene acetal, cyclohexylidene acetal, benzylidene acetal, p-methoxybenzylidene acetal and o,p-dimethoxybenzylidene acetal, acyclic acetals, such as tetrahydropyranyl (Thp), methoxymethyl (MOM), methoxyethoxymethyl (MEM), benzyloxymethyl (BOM) and methylthiomethyl (MTM). Particularly preferred hydroxyl-protecting groups are benzyl, acetyl, tert-butyl and TBS.

The liberation of the compounds of the formula I from their functional derivatives is known from the literature for the protecting group used in each case (for example T. W. Greene, P. G. M. Wuts, Protective Groups in Organic Chemistry, 2nd Edn., Wiley, New York 1991 or P. J. Kocienski, Protecting Groups, 1 st Edn., Georg Thieme Verlag, Stuttgart—New York, 1994). Use may also be made here of variants which are known per se, but are not mentioned here in greater detail.

The groups BOC and O-tert-butyl may, for example, be removed preferentially using TFA in dichloromethane or using approximately 3 to 5N HCl in dioxane at 15-30° C., and the Fmoc group using an approximately 5 to 50% solution of dimethylamine, diethylamine or piperidine in DMF at 15-30° C. The Aloc group can be removed under gentle conditions with noble-metal catalysis in chloroform at 20-30° C. A preferred catalyst is tetrakis(triphenylphosphine)palladium(0).

Some of the starting compounds of the formulae III, IV, VI, VII, IX and X are known. For example, compounds of the formula IX are prepared as described in WO 0048996 A2. If they are novel, however, they can be prepared by methods known per se.

Compounds of the formula V are obtained by a peptide-analogous coupling of the compounds of the formula III with a compound of the formula IV or by peptide-analogous coupling of the compounds of the formula VI with a compound of the formula VII under standard conditions.

The conversion of the compounds V into the compounds of the formula II can be carried out, for example, with triethylammonium chloride and an azide, in particular sodium azide, under standard conditions. A suitable methods is described, for example, in The Chemistry of Heterocycles, Georg Thieme Verlag, Stuttgart 1995.

Compounds of the formula VIII are obtained by condensation of compounds of the formula IX with compounds of the formula X, preferably in the presence of a dehydrating agent, for example a carbodiimide, such as dicyclohexylcarbodiimide (DCC), N-(3-dimethylaminopropyl)-N′-ethylcarbodiimide hydrochloride (EDC) or diisopropylcarbodiimide (DIC), furthermore, for example, propanephosphonic anhydride.

Conventional methods for the preparation of acylsulfonamides are described, for example, in Pelletier, J. C., Hesson, D. P., Synlett, 1995, 11, 1141-1142 or Barraclough, P., Caldwell, A. G., J. Chem. Soc., Perkin Trans 1, 1989, 181.

Conventional methods of peptide synthesis are described, for example, in Houben-Weyl, 1.c., Volume 15/II, 1974, pages 1 to 806.

The coupling reaction preferably succeeds in the presence of a dehydrating agent, for example a carbodiimide, such as dicyclohexylcarbodiimide (DCC), N-(3-dimethylaminopropyl)-N′-ethylcarbodiimide hydrochloride (EDC) or diisopropylcarbodiimide (DIC), furthermore, for example, propanephosphonic anhydride (cf. Angew. Chem. 1980, 92, 129), diphenylphosphoryl azide or 2-ethoxy-N-ethoxycarbonyl-1,2-dihydroquinoline, in an inert solvent, for example a halogenated hydrocarbon, such as dichloromethane, an ether, such as tetrahydrofuran or dioxane, an amide, such as DMF or dimethylacetamide, a nitrile, such as acetonitrile, in dimethyl sulfoxide or in the presence of this solvent, at temperatures between about −10 and 400, preferably between 0 and 300. The reaction time, depending on the conditions used, is between a few minutes and several days.

It has proven particularly advantageous to add the coupling reagent TBTU (O-(benzotriazol-1-yl)-N,N,N′,N′-tetramethyluronium tetrafluoroborate) or O-(benzotriazol-1-yl)-N,N,N′,N′-tetramethyluronium hexafluorophosphate, since in the presence of one of these compounds only slight racemisation occurs and no cytotoxic by-products are formed.

Instead of compounds of the formula IV and/or VII, it is also possible to employ derivatives of compounds of the formula IV and/or VII, preferably a pre-activated carboxylic acid, or a carboxylic acid halide, a symmetrical or mixed anhydride or an active ester. Radicals of this type for activation of the carboxyl group in typical acylation reactions have been described in the literature (for example in the standard works, such as Houben-Weyl, Methoden der organischen Chemie [Methods of Organic Chemistry], Georg-Thieme-Verlag, Stuttgart). Activated esters are advantageously formed in situ, for example by addition of HOBt (1-hydroxybenzotriazole) or N-hydroxysuccinimide.

The reaction is generally carried out in an inert solvent; if a carboxylic acid halide is used, it is carried out in the presence of an acid-binding agent, preferably an organic base, such as triethylamine, dimethylaniline, pyridine or quinoline.

The addition of an alkali or alkaline-earth metal hydroxide, carbonate or bicarbonate or another salt of a weak acid of the alkali or alkaline-earth metals, preferably of potassium, sodium, calcium or caesium, may also be favourable.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, benzenesulfonic acid, trimethoxybenzoic acid, adamantanecarboxylic acid, p-toluenesulfonic acid, glycolic acid, embonic acid, chlorophenoxyacetic acid, aspartic acid, glutamic acid, proline, glyoxylic acid, palmitic acid, para-chlorophenoxyisobutyric acid, cyclohexanecarboxylic acid, glucose 1-phosphate, naphthalenemono- and -disulfonic acids or laurylsulfuric acid. Salts with physiologically unacceptable acids, for example picrates, can be used to isolate and/or purify the compounds of the formula I.

On the other hand, compounds of the formula I can be converted into the corresponding metal salts, in particular alkali metal salts or alkaline earth metal salts, or into the corresponding ammonium salts, using bases (for example sodium hydroxide, potassium hydroxide, sodium carbonate or potassium carbonate).

The invention also relates to the compounds of the formula I according to claim 1, stereoisomers thereof and physiologically acceptable salts or solvates thereof as medicament active ingredients.

The invention furthermore relates to compounds of the formula I according to claim 1, stereoisomers thereof and physiologically acceptable salts or solvates thereof as integrin receptor antagonists.

The invention also relates to the compounds of the formula I according to claim 1, stereoisomers thereof and physiologically acceptable salts or solvates thereof for use in combating diseases.

The invention furthermore relates to pharmaceutical preparations comprising at least one compound of the formula I, stereoisomers thereof and/or one of its physiologically acceptable salt or solvates. To this end, the compounds of the formula I can be brought into a suitable dosage form together with at least one solid, liquid and/or semiliquid excipient or adjuvant and, if desired, in combination with one or more further active ingredients.

The invention therefore likewise relates to the use of compounds of the formula I, stereoisomers thereof and/or physiologically acceptable salts or solvates thereof for the preparation of a medicament.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oily or aqueous solutions, furthermore suspensions, emulsions or implants, and suitable for topical application are ointments, creams or powders. The novel compounds can also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, dyes, flavours and/or a plurality of further active ingredients, for example one or more vitamins.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronised form, in which case one or more additional physiologically acceptable solvents may be present, for example ethanol. Inhalation solutions can be administered with the aid of conventional inhalers.

The compounds of the formula I, stereoisomers thereof and/or physiologically acceptable salts thereof can be employed as medicament active ingredients in human and veterinary medicine, in particular for the prophylaxis and/or therapy of circulation disorders, pulmonary fibrosis, pulmonary embolism, thrombosis, in particular deep-vein thrombosis, cardiac infarction, arteriosclerosis, aneurysma dissecans, transient ischaemic attacks, apoplexia, angina pectoris, in particular unstable angina pectoris, tumour diseases, such as tumour development or tumour metastasis, osteolytic diseases, such as osteoporosis, hyperparathyroidism, Paget's disease, malign hypercalcaemia, incompatible blood transfusion, pathologically angiogenic disorders, such as, for example, inflammation, ophthalmological disorders, diabetic retinopathy, macular degeneration, myopia, corneal transplant, ocular histoplasmosis, rheumatic arthritis, osteoarthritis, rubeotic glaucoma, ulcerative colitis, Crohn's disease, atherosclerosis, psoriasis, restenosis, in particular after angioplasty, multiple sclerosis, pregnancy, absumptio placentaris, viral infection, bacterial infection, fungal infection, foot and mouth disease, in the case of acute kidney failure and in the case of wound healing for supporting the healing process. Particular preference is given to use for the treatment of tumour diseases, osteolytic diseases, in particular osteoporosis, and for the treatment of restenosis after angioplasty.

The substances according to the invention are preferably administered in doses of from about 0.05 to 500 mg, in particular from 0.5 to 100 mg, per dosage unit. The daily dose is preferably from about 0.01 to 2 mg/kg of body weight. However, the specific dose for each patient depends on a wide variety of factors, for example on the efficacy of the specific compound employed, on the age, body weight, general state of health, sex, on the diet, on the time and method of administration, on the rate of excretion, medicament combination and severity of the particular disease to which the therapy applies. Parenteral administration is preferred.

The compounds of the formula I have one or more centres of chirality and can therefore exist in racemic or optically active form. Racemates obtained can be resolved into the enantiomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid, and the various optically active camphorsulfonic acids, such as β-camphorsulfonic acid. Resolution of the enantiomers with the aid of a column filled with an optically active resolving agent (for example dinitrobenzoylphenylglycine) is also advantageous; an example of a suitable eluent is a mixture of hexane/isopropanol/acetonitrile, for example in the volume ratio 82:15:3.

It is of course also possible to obtain optically active compounds of the formula I by the methods described above by using starting materials which are already optically active.

Above and below, all temperatures are given in ° C. In the following examples, “conventional work-up” means that, if necessary, water is added, if necessary, depending on the constitution of the end product, the pH is adjusted to a value between 2 and 10, the mixture is extracted with ethyl acetate or dichloromethane, the phases are separated, the organic phase is dried over sodium sulfate and evaporated, and the product is purified by chromatography on silica gel, by preparative HPLC and/or by crystallisation. The purified compounds are, if desired, freeze-dried.

The eluents used are gradients of acetonitrile (B) with 0.08% of TFA (trifluoroacetic acid) and water (A) with 0.1% of TFA. The gradient is indicated in percent by volume of acetonitrile.

The HPLC analyses (retention time RT) were carried out in the following systems:

3 μm Silica-Rod column with a 210 second gradient from 20 to 100% water/acetonitrile/0.01% trifluoroacetic acid, at a flow rate of 2.2 ml/min and with detection at 220 nm.

The compounds purified by preparative HPLC are isolated as trifluoroacetates.

Mass spectrometry (MS) by means of FAB (fast atom bombardment): MS-FAB (M+H)⁺.

The examples explain the invention without the latter being restricted thereto.

If the compounds described as examples are able to exist as various stereoisomers and no stereochemical data are given, mixtures of the stereoisomers are present in each case.

logD values given above and below are partition coefficients of the compounds in question octanol/water at a pH of 7.4 (logD_{(7.4)}).

### EXAMPLE 1

Preparation of N-[1-biphenyl-4-yl-2-(1H-tetrazol-5-yl)ethyl]-2-{3-[3-(pyridin-2-ylamino)propyl]ureido}acetamide

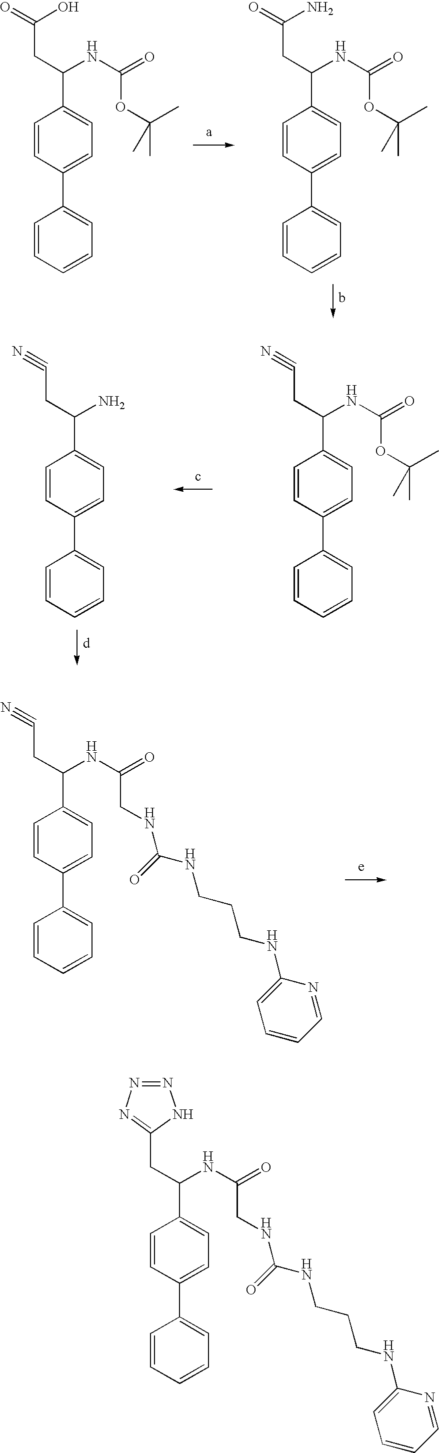

a 0.751 g of 3-biphenyl-4-yl-3-tert-butoxycarbonylaminopropionic acid and 0.310 ml of triethylamine are initially introduced in 20 ml of tetrahydrofuran (THF), 0.209 ml of ethyl chloroformate is added dropwise, and the mixture is stirred for 20 minutes. 0.412 ml of 25% ammonia is subsequently added, and the mixture is cooled to room temperature and stirred for a further 20 minutes. The reaction product in crystalline form is filtered off with suction and dried in a vacuum drying cabinet, giving 3-biphenyl-4-yl-3-tert-butoxycarbonylaminopropionamide.

b 0.700 g of 3-biphenyl-4-yl-3-tert-butoxycarbonylaminopropionamide and 0.582 ml of triethylamine are initially introduced in 20 ml of THF, 0.306 ml of triflouroacetic anhydride is added dropwise with ice-cooling, and the mixture is allowed to cool to room temperature. After 1 hour, 2 ml of water are added, the mixture is evaporated to dryness, the resultant residue is dissolved in ethyl acetate and water, and the aqueous phase is washed twice with ethyl acetate. The combined organic phases are washed with 0.1 N HCl and 0.1 N NaOH, dried and evaporated to dryness, giving tert-butyl (1-biphenyl-4-yl-2-cyanoethyl)carbamate.

c 5 ml of triflouroacetic acid (TFA) are added to 0.350 g of tert-butyl (1-biphenyl-4-yl-2-cyanoethyl)carbamate dissolved in 10 ml of dichloromethane (DCM), and the mixture is left to stand for 30 minutes and evaporated to dryness, giving 3-amino-3-biphenyl-4-ylpropionitrile.

d 0.233 g of 3-amino-3-biphenyl-4-ylpropionitrile, 267.4 mg of {3-[3-(pyridin-2-ylamino)propyl]ureido}acetic acid, 15 ml of N,N-dimethylformamide, 0.340 g of TBTU and 0.195 ml of DIPEA are stirred overnight at room temperature as a mixture and subsequently evaporated to dryness, and the residue obtained is purified over a silica-gel column (eluent: ethyl acetate/methanol 9:1). The substance was washed from the column using methanol and separated by preparative HPLC, giving N-(1-biphenyl-4-yl-2-cyanoethyl)-2-{3-[3-(pyridin-2-ylamino)propyl]ureido}acetamide.

e 20.0 mg of N-(1-biphenyl-4-yl-2-cyanoethyl)-2-{3-[3-(pyridin-2-yl-amino)propyl]ureido}acetamide, 5.0 ml of N,N-dimethylformamide, 0.012 g of sodium azide and 0.025 of triethylammonium chloride are stirred for 6 hours at 80° C. as a mixture, filtered with suction, evaporated to dryness and purified by preparative HPLC, giving N-[1-biphenyl-4-yl-2-(1H-tetrazol-5-yl)ethyl]-2-{3-[3-(pyridin-2-ylamino)propyl]ureido}acetamide, triflouroacetate (EMD 387 890), RT 1.058 min, logD_{(7.4)}+6.19, FAB-MS (M+H)⁺500.15.

### EXAMPLE 2

Preparation of N-[1-biphenyl-4-yl-2-(1H-tetrazol-5-yl)ethyl]-2-[5-(pyridin-2-yl-amino)pentanoylamino]acetamide

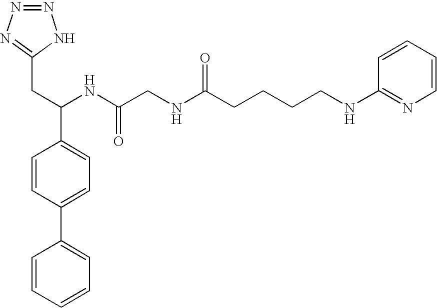

a 0.398 g of 3-biphenyl-4-yl-3-(2-tert-butoxycarbonylaminoacetylamino)propionic acid and 0.139 ml of triethylamine are initially introduced in 20 ml of tetrahydrofuran (THF), 0.095 ml of ethyl chloroformate is added dropwise, and the mixture is stirred for 20 minutes. 0.082 ml of 25% ammonia is subsequently added, and the mixture is cooled to room temperature and stirred for a further 3 hours. The reaction product in crystalline form is filtered off with suction and dried in a vacuum drying cabinet, giving tert-butyl [(1-biphenyl-4-yl-2-carbamoylethylcarbamoyl)methyl]carbamate.

b 0.360 g of tert-butyl [(1-biphenyl-4-yl-2-carbamoylethylcarbamoyl)methyl]carbamate and 0.277 ml of triethylamine are initially introduced in 20 ml of THF, 0.139 ml of triflouroacetic anhydride is added dropwise with ice-cooling, and the mixture is allowed to cool to room temperature. After 1 hour, 2 ml of water are added, the mixture is evaporated to dryness, the residue obtained is dissolved in ethyl acetate and water, and the aqueous phase is washed twice with ethyl acetate. The combined organic phases are washed with 0.1 N HCl and 0.1 N NaOH, dried and evaporated to dryness, giving N-(1-biphenyl-4-yl-2-cyanoethyl)-2-(tert-butoxycarbonylamino)acetamide.

c Trifluoroacetic acid (TFA) 1:1 is added to 0.190 g of N-(1-biphenyl-4-yl-2-cyanoethyl)-2-(tert-butoxycarbonylamino)acetamide in dichloromethane (DCM), and the mixture is stirred for 15 minutes and evaporated to dryness, giving 2-amino-N-(1-biphenyl-4-yl-2-cyanoethyl)acetamide.

d 0.190 g of 2-amino-N-(1-biphenyl-4-yl-2-cyanoethyl)acetamide, 0.326 ml of 5-(pyridin-2-ylamino)pentanoic acid, 2.5 ml of N,N-dimethylformamide, 2.5 ml of 1,2-dichloroethane 0.100 g of 4-(diethylamino)pyridine and 0.365 g of N-cyclohexylcarbodiimide, N′-methylpolystyrene HL are stirred overnight at room temperature as a mixture, 2.0 g of a strongly acidic ion exchanger are added, and the mixture is stirred for 2 hours, filtered with suction and separated by preparative HPLC, giving N-(1-biphenyl-4-yl-2-cyanoethyl)-2-[5-(pyridin-2-ylamino)pentanoylamino]acetamide.

e 0.041 g of N-(1-biphenyl-4-yl-2-cyanoethyl)-2-[5-(pyridin-2-ylamino)pentanoylamino]acetamide, 3.0 ml of N,N-dimethylformamide, 0.059 g of sodium azide and 0.124 g of triethylammonium chloride are stirred for 6 hours at 80° C. as a mixture, filtered with suction, evaporated to dryness and purified by preparative HPLC, giving N-[1-biphenyl-4-yl-2-(1H-tetrazol-5-yl)ethyl]-2-[5-(pyridin-2-ylamino)pentanoylamino]-acetamide, triflouroacetate (EMD 389 889), RT 1.296 min, logD_{(7.4)}+6.52, FAB-MS (M+H)⁺499.

### EXAMPLE 3

N-[1-biphenyl-4-yl-2-(methanesulfonylaminocarbonyl)ethyl]-2-[5-(pyridin-2-ylamino)pentanoylamino]acetamide

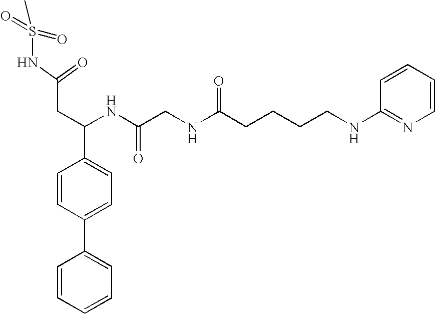

0.095 g of 3-biphenyl-4-yl-3-{2-[5-(pyridin-2-ylamino)pentanoylamino]-acetylamino}propionic acid, 0.019 g of methanesulfonamide, 0.115 g of N-cyclohexylcarbodiimide, N′-methyl-polystyrene HL, 0.080 g of 4-(dimethylamino)pyridine, 2.0 ml of 1,2-dichloroethane and 2.0 ml of tert-butanol are stirred together at 50° C. for 24 hours. 1.5 g of strongly acidic ion exchanger are subsequently added, and the mixture is stirred for 2 hours, filtered with suction and evaporated to dryness, giving N-[(1-biphenyl-4-yl-2-(methanesulfonylaminocarbonyl)ethyl]-2-[5-(pyridin-2-yl-amino)pentanoylamino]acetamide, triflouroacetate (EMD 387 803), RT 1.298 min, logD_{(7.4)}−0.38, FAB-MS (M+H)⁺552.

The examples below relate to pharmaceutical preparations:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

### EXAMPLE I

### Inhalation Spray

14 g of active ingredient of the formula I are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14 mg.

1. Compounds of the formula I 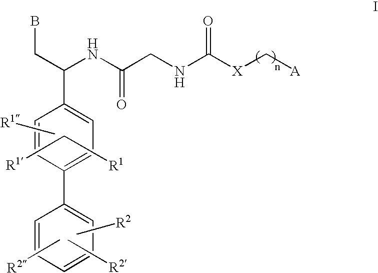
in which
A is NH₂, —(HN═)C—NH₂, —NH—C(═NH)—NH₂, A′-C(═NH)—NH—, Het¹- or Het¹-NH—, where the primary amino groups may also be provided with conventional amino-protecting groups,
B is tetrazolyl or an alkylsulfonylaminocarbonyl group,
R is H, A′, C₆-C₁₄-cycloalkyl, C₆-C₁₀-aryl or C₇-C₁₄-aralkyl, which may be monosubstituted or polysubstituted by R³ and whose alkyl carbon chain may be interrupted by 0,
R¹, R^{1″} and R^{1″}, independently of one another, are H, F, Cl, Br, 1, NO₂, NH₂, NHR, NRR, OH, OR, CO—R, SO₃R, SO₂R or SR,
R², R^{2′} and R^{2″}, independently of one another, are H, F, Cl, Br, 1, NO₂, NH₂, NHR, NRR, OH, OR, CO—R, SO₃R, SO₂R or SR,
R³ is F, Cl, Br, I, NO₂, CF₃, OH, CN, OCF₃, SCF₃, methoxy or ethoxy,
Het¹ is a monocyclic or bicyclic heterocyclic radical having from 1 to 4 N atoms, which may be unsubstituted or monosubstituted or disubstituted by A′, NHA′, NA′₂ and/or NH₂,
A′ is alkyl having from 1 to 8 carbon atoms,
X is nothing, O, NH or CH₂,
n is 2, 3 or 4,
stereoisomers thereof and physiologically acceptable salts and solvates thereof.

2. Compounds according to claim 1, characterised in that these are
N-[1-biphenyl-4-yl-2-(1H-tetrazol-5-yl)ethyl]-2-[5-(pyridin-2-ylamino)pentanoylamino]acetamide,
N-[1-biphenyl-4-yl-2-(1H-tetrazol-5-yl)ethyl]-2-{3-[3-(pyridin-2-ylamino)propyl]ureido}acetamide,
N-[1-biphenyl-4-yl-2-(methanesulfonylaminocarbonyl)ethyl]-2-[5-(pyridin-2-ylamino)pentanoylamino]acetamide.

3. Process for the preparation of the compounds of the formula II 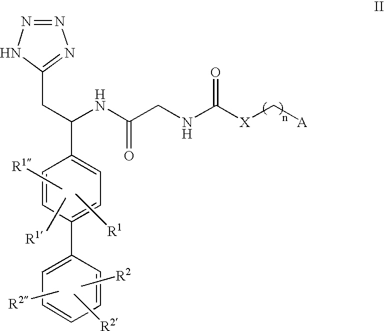
according to claim 1, stereoisomers thereof and salts and solvates thereof, in which A, X, R¹, R^{1′}, R^{1″}, R², R^{2′}, R^{2″} and n are as defined in the formula I,
characterised in that
(a) a compound of the formula III 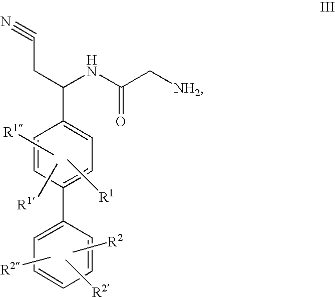
in which R¹, R^{1′}, R^{1″}, R², R^{2′} and R^{2″} are as defined in the formula I and in which, in the case where R¹, R^{1′}, R^{1″}, R², R^{2′} and/or R^{2″} contain free hydroxyl and/or amino groups, these are protected by a protecting group,
is reacted with a compound of the formula IV 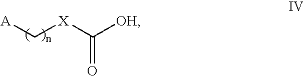
in which A and n are as defined in the formula I, and X is nothing or —(CH₂)—, and in which, in the case where A contains amino groups, these are each protected by protecting groups,
to give a compound of the general formula V 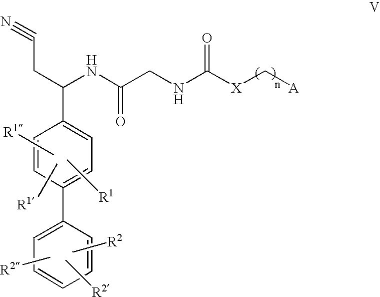
in which A, R¹, R^{1′}, R^{1″}, R², R^{2′}, R^{2″} and n are as defined in the formula I and X is —(CH₂)—,
and the resultant compound of the formula V is subsequently converted into a compound of the above formula II, in which R¹, R^{1′}, R^{1″}, R², R^{2′}, R^{2″}, A and n are as defined therein, and X is —(CH₂)—,
and, if desired, the protecting groups present on A, R¹, R^{1′}, R^{1″}2, R², R^{2′} and/or R^{2″} are removed, or
(b) a compound of the formula VI 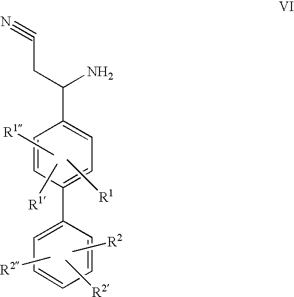
in which R¹, R^{1′}, R^{1″}, R², R^{2′} and R^{2″} are as defined in the formula I and in which, in the case where R¹, R^{1′}, R^{1″}, R², R^{2′} and/or R^{2″} contain free hydroxyl and/or amino groups, these are protected by a protecting group,
is reacted with a compound of the formula VII 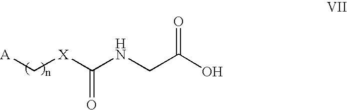
in which A, X and n are as defined in the formula I and in which, in the case where A contains free amino groups, these are each protected by protecting groups,
to give a compound of the above general formula V, in which R¹, R^{1′}, R^{1″}, R², R^{2′}, R^{2″}, A and n are as defined therein, and X is as defined in the formula I,
and the resultant compound of the formula V is subsequently converted into a compound of the above general formula II, in which R¹, R^{1′}, R^{1″}, R², R^{2′}, R^{2″}, A, X and n are as defined therein, and, if desired, the protecting groups present on A, R¹, R^{1′}, R^{1″}, R², R^{2′} and/or R^{2″} are removed, or
(c) one or more of the radicals R¹, R^{1′}, R^{1″}, R², R^{2′} and/or R^{2″} in a compound of the formula II, in which R¹, R^{1′}, R^{1″}, R², R^{2′}, R^{2″}, A, X and n are as defined therein, are converted into one or more radicals R¹, R^{1′}, R^{1″}, R², R^{2′} and/or R^{2″} by, for example,
i) alkylating a hydroxyl group or
ii) alkylating an amino group, and/or
a basic or acidic compound of the formula II is converted into one of its salts or solvates by treatment with an acid or base.

4. Process for the preparation of compounds of the formula VIII 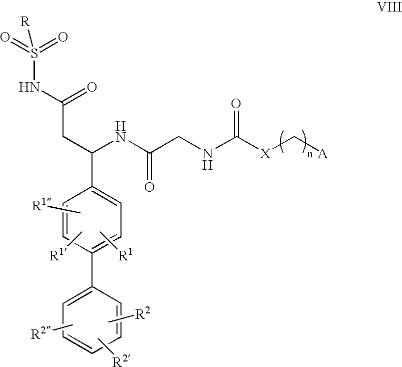
according to the formula I, salts and solvates thereof, in which A, A′, X, R, R¹, R^{1′}, R^{1″}, R², R^{2′}, R^{2″} and n are as defined in the formula I, characterised in that
(a) a compound of the formula IX 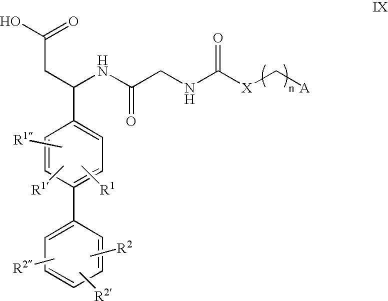
in which A, X, R¹, R^{1′}, R^{1″}, R², R^{2′}, R^{2″} and n are as defined in the formula I, and in which, in the case where A, R¹, R^{1′}, R^{1″}, R², R^{2′} and/or R^{2″} contain free hydroxyl or amino groups, these are protected by a protecting group,
is reacted with a compound of the formula X 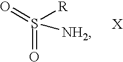
in which R is as defined in the formula I,
to give a compound of the above-mentioned general formula VIII, in which A, X, R, R¹, R^{1′}, R^{1″}, R², R^{2′}, R^{2″} and n are as defined therein,
and, if desired, the protecting groups present on A, R¹, R^{1′}, R^{1″}, R², R^{2′} and/or R^{2″} are removed, or
(b) one or more radicals R¹, R^{1′}, R^{1″}, R², R^{2′} and/or R^{2″} in a compound of the formula VIII are converted into one or more radicals R¹, R^{1′}, R^{1″}, R², R^{2′} and/or R^{2″} by, for example,
i) alkylating a hydroxyl group or
ii) alkylating an amino group, and/or
a basic or acidic compound of the formula VIII is converted into one of its salts or solvates by treatment with an acid or base.

5. Compounds of the formula I according to claim 1 or 2, stereoisomers thereof and physiologically acceptable salts or solvates thereof as medicament active ingredients.

6. Compounds of the formula I according to claim 1 or 2, stereoisomers thereof and physiologically acceptable salts or solvates thereof as integrin receptor inhibitors.

7. Compounds of the formula I according to claim 1 or 2, stereoisomers thereof and physiologically acceptable salts or solvates thereof for use in combating diseases.

8. Medicament, characterised in that it comprises at least one compound of the formula I according to claim 1 or 2, stereoisomers thereof and/or one of its physiologically acceptable salts or solvates.

9. Use of compounds of the formula I according to claim 1 or 2, stereoisomers thereof and/or physiologically acceptable salts or solvates thereof for the preparation of a medicament.

10. Use of compounds of the formula I according to claim 1 or 2, stereoisomers thereof and/or physiologically acceptable salts or solvates thereof for the preparation of a medicament for the prophylaxis and/or therapy of circulation disorders, pulmonary fibrosis, pulmonary embolism, thrombosis, in particular deep-vein thrombosis, cardiac infarction, arteriosclerosis, aneurysma dissecans, transient ischaemic attacks, apoplexia, angina pectoris, in particular unstable angina pectoris, tumour diseases, such as tumour development or tumour metastasis, osteolytic diseases, such as osteoporosis, hyperparathyroidism, Paget's disease, malign hypercalcaemia, incompatible blood transfusion, pathologically angiogenic disorders, such as, for example, inflammation, ophthalmological disorders, diabetic retinopathy, macular degeneration, myopia, corneal transplant, ocular histoplasmosis, rheumatic arthritis, osteoarthritis, rubeotic glaucoma, ulcerative colitis, Crohn's disease, atherosclerosis, psoriasis, restenosis, in particular after angioplasty, multiple sclerosis, absumptio placentaris, viral infection, bacterial infection, fungal infection, acute kidney failure and wound healing.
